(19) United States
(12) Patent Application Publication
(10) Pub. No.: US 2022/0401736 A1
(43) Pub. Date: Dec. 22, 2022

(54) APPARATUSES, SYSTEMS AND METHODS FOR IMPLANTABLE STIMULATOR WITH EXTERNALLY TRAINED CLASSIFIER

(71) Applicant: University of Washington, Seattle, WA (US)

(72) Inventors: Jeffrey Andrew Herron, Seattle, WA (US); Benjamin Isaac Ferleger, Seattle, WA (US); Howard Jay Chizeck, Seattle, WA (US); Andrew L. Ko, Seattle, WA (US)

(73) Assignee: University of Washington, Seattle, WA (US)

(21) Appl. No.: 17/656,156

(22) Filed: Mar. 23, 2022

Related U.S. Application Data
(60) Provisional application No. 63/213,358, filed on Jun. 22, 2021.

(51) Int. Cl. A61N 1/36; A61N 1/05

(52) U.S. Cl. CPC A61N 1/36139; A61N 1/0534

(57) ABSTRACT

Embodiments of the disclosure are drawn to implantable stimulator with machine learning based classifier. An implantable system includes sensors which provide sensor information to an implantable unit. The implantable unit uses a classifier on the sensor information to select a stimulation procedure which is applied via a stimulation electrode. The classifier may be generated by a trained machine learning model. The classifier may be trained on an external unit which is not implanted in the subject. The classifier may be trained based on sensor information from the implanted sensors as well as symptom information.

### CROSS-REFERENCE TO RELATED APPLICATIONS

This application claims the benefit under 35 § U.S.C. 119 of the earlier filing date of U.S. Provisional Application Ser. No. 63/213,358 filed Jun. 22, 2021, the entire contents of which is hereby incorporated by reference, in its entirety, for any purpose.

### STATEMENT REGARDING RESEARCH & DEVELOPMENT

This invention was made with government support under Grant No. EEC-1028725, awarded by the National Science Foundation. The government has certain rights in the invention.

### BACKGROUND

Essential tremor (ET) is the world's most common type of movement disorder (MD), affecting an estimated 4.6% of the population over age 65. ET is characterized by its cardinal symptoms of kinetic and postural upper limb tremor, as opposed to the rest tremor common in other conditions. Deep brain stimulation (DBS) of the ventral intermediate nucleus (VIM) of the thalamus is an established treatment for ET. Follow up studies have demonstrated that DBS is a safe and effective treatment for ET, with most adverse effects related to perioperative complications and occurring at low rates. At present, continuous, or conventional, DBS (cDBS), wherein stimulation parameters are set by a clinician and left continuously at those levels, is the standard of clinical care.

Nonetheless, cDBS remains an imperfect treatment. For example, the implantable pulse generator (IPG) battery may only be replaced through revision surgeries that must be conducted every few years. Several side effects, including paresthesia, difficulty speaking, balance issues, and sexual emotional disinhibition, are associated with cDBS treatment.

### SUMMARY

in at least one aspect, the present disclosure relates to a method which includes receiving sensor data from implanted sensors at an external unit, classifying the sensor data based on symptom information, training a machine learning model to generate a classifier based on the classified sensor data, and loading the classifier onto an implantable unit.

The method may also include selecting a stimulation procedure of the implantable unit based on the sensor data from the implanted sensors and the classifier, and providing stimulation to a subject from the implantable unit based on the selected stimulation procedure. The method may also include selecting a first stimulation procedure based on a first result from the classifier and selecting a second stimulation procedure based on a second result from the classifier.

The method may also include obtaining the symptom information from an additional sensor. The additional sensor may be placed externally on the subject. The external unit may include one or more networked devices in a cloud computing system.

The method may also include collecting a first set of sensor data while the subject is at rest, and a second set of sensor data while the patient is active and training the classifier to determine if the subject is at rest or if the subject is active based on the first set of sensor data and the second set of sensor data. The first set of sensor data may include a first portion where the implantable unit is providing active stimulation and a second portion Where the implantable unit is not providing active stimulation. The second set of sensor data may include a third portion where the implantable unit is providing active stimulation and a fourth portion where the implantable unit is not providing active stimulation. The method may include biasing the classifier.

In at least one aspect, the present disclosure may relate to a system which includes an implantable unit implanted in a subject and an external unit. The implantable unit includes implanted sensors configured to provide sensor information, a stimulation electrode, a processor, and a memory. The memory is loaded with non-transitory instructions, which, when executed by the processor cause the implantable unit to select a stimulation procedure based on the sensor information and a classifier and apply stimulation to the stimulation electrode based on the selected stimulation procedure. The external unit includes a processor and a memory loaded with non-transitory instructions which, when executed by the processor cause the external unit to train the classifier based on data from the sensors and symptom information and load the classifier onto the memory of the implantable unit.

The implantable unit may be an adaptive deep brain stimulation (aDBS) system. The implantable sensors may include electrocorticography (ECoG) strips which collect local field potential (LFP) information.

The memory of the external unit may include instructions which, when executed by the processor of the external unit, cause the external unit to train the classifier to determine active or at rest state of subject. The memory of the implantable unit may include instructions which, when executed by the processor of the implantable unit, cause the implantable unit to select a first stimulation procedure when the classifier determines that the subject is active and select a second stimulation procedure when the classifier determines that the subject is at the rest state. The classifier may be biased to preferentially select the active state based on the sensor information.

The symptom information may include labels for sensor information collected during different periods of subject activity. The external unit may include one or more networked systems in a location remote from the implantable unit. The stimulation electrode may be a deep brain stimulation electrode implanted in the subject's nervous system. The system may further include a wearable sensor placed on the subject, wherein the symptom information is based, in part, on information from the wearable sensor.

In at least one aspect, the present disclosure relates to an apparatus including implanted sensors which provide sensor information, a stimulation electrode, and an implantable unit. The implantable unit operates to classify the sensor information based on a classifier, select a stimulation procedure based on the classified sensor information and provide stimulation via the stimulation electrode based on a selected stimulation procedure, The classifier is trained by a machine learning algorithm, and the implantable unit applies stimulation with the stimulation electrode based on the selected stimulation procedure.

The classifier may be trained on an external unit which is not implanted in the subject. The classifier may be trained based on the sensor information from the implantable sensors and information from the stimulation electrode. The implanted sensors may include an electrocorticography (ECoG) strip.

The classifier may determine if a subject is at an active state or a rest state. The implantable unit may provide stimulation with the stimulation electrode when the classifier determines the active state and may not provide stimulation with the stimulation electrode when the classifier determines the rest state. The implanted sensors, the stimulation electrode, and the implantable unit may be components of an adaptive deep brain stimulation (aDBS) system.

### BRIEF DESCRIPTION OF THE DRAWINGS

FIG. 1 is a block diagram of a distributed training system for an implantable device according to some embodiments of the present disclosure.

FIG. 2 is a flow chart of a method of training a classifier for an implantable system according to some embodiments of the present disclosure.

FIG. 3 is a flow chart of a method of providing stimulation with an implantable system according to some embodiments of the present disclosure.

FIG. 4 shows a set of graphs which represent example training data according to some embodiments of the present disclosure.

FIG. 5 shows graphs of example distributions of measured data and biased classification thresholds according to some embodiments of the present disclosure.

FIG. 6 is a set of graphs showing results of an implantable system using a classifier according to some embodiments of the present disclosure.

FIGS. 7A and 713 are block diagrams of an example computing network and computing device according to some embodiments of the present disclosure.

### DETAILED DESCRIPTION

The following description of certain embodiments is merely exemplary in nature and is in no way intended to limit the scope of the disclosure or its applications or uses. In the following detailed description of embodiments of the present systems and methods, reference is made to the accompanying drawings which form a part hereof, and which are shown by way of illustration specific embodiments in which the described systems and methods may be practiced. These embodiments are described in sufficient detail to enable those skilled in the art to practice presently disclosed systems and methods, and it is to be understood that other embodiments may be utilized and that structural and logical changes may be made without departing from the spirit and scope of the disclosure. Moreover, for the purpose of clarity, detailed descriptions of certain features will not be discussed when they would be apparent to those with skill in the art so as not to obscure the description of embodiments of the disclosure. The following detailed description is therefore not to be taken in a limiting sense, and the scope of the disclosure is defined only by the appended claims.

Deep brain stimulation (DBS) is a safe and established treatment for essential tremor (ET) and several other movement disorders. One approach to ameliorating the concerns associated with cDBS is adaptive DBS (aDBS), interchangeably referred to as closed-loop DBS, in which stimulation parameters are modulated in real time based on biofeedback from either external or implanted sensors. Using external or implanted sensors to provide biofeedback, aDBS uses inferences about the patient's state extracted from this sensed data to modulate stimulation parameters and reduce overall stimulation. This may reduce battery drain while potentially reducing the incidence and severity of side effects. The nature of ET among conditions for which DBS is prescribed—specifically, the predictable and activity-dependent nature of symptom manifestation may make it well-suited as a testbed for the investigation of aDBS strategies.

Most previously investigated aDBS systems have made use of either external sensing methods or distributed data processing structures. External sensing, such as inertial measurement unit (IMU) data (e.g., accelerometer data) from a smartwatch affixed to the patient's treated wrist, provides easily defined and understood real-time feedback on symptom severity and patient activity. Distributed data processing structures use data from implanted sensors, such as local field potential (LFP) data recorded using electrocorticography (ECoG) strips, in order to infer the patient's state through the deployment of machine learning algorithms on an external experimental computer or other computational device. The use of a distributed system may allow the application of data processing and machine learning techniques on noisy, non-stationary neural data. This may permit the development of highly accurate algorithms to predict patient state.

Previously tested systems, however, have drawbacks, and fallen short of translational applicability due to, for example, the requirement for patients to continuously wear the necessary sensors or processing devices, as well as privacy and security concerns.

With respect to sensors, external sensing systems require a patient to wear some symptom-tracking device at virtually all times, which is unlikely to serve in a translational capacity due to the likelihood of patients either forgetting the required device some days, or even their unwillingness to wear it altogether. Likewise, distributed systems tether patients to an associated data processing device, limiting mobility in cases where the tether is a physical wire and, with wireless systems, running into the same translational problems encountered with wearable devices.

Additionally, with respect to privacy and security, both wearable and distributed systems raise clear privacy and security concerns due to the streaming of protected personal information and, perhaps more concerning, the increased potential for malicious third-party interference with the device itself introduced by this streaming.

Other previously tested systems include fully implanted systems in which internally detected biomarkers are processed in the IPG itself and an on-board algorithm applied to modulate stimulation parameters in real time. Such system would minimize concerns about a patient remembering their external devices by making the process entirely internal and automated, with the added benefit of intrinsically resolving issues of privacy and security during chronic treatment. However, these systems too have their drawbacks. For example, due to the necessity of using only internally recorded data and the limited processing power available on implanted devices relative to modern computers and mobile devices, these systems would lack the ease of interpretability in programming ensured by wearable systems and the sheer processing power available to distributed systems. Such drawbacks have limited the translational applicability of fully implanted aDBS,

Examples of technology described herein includes systems, apparatuses, and methods for implantable stimulators with externally trained classifiers. A subject, for example a patient with ET, has an implantable unit, such as an IPG, which provides stimulation via one or more electrodes implanted in the subject's nervous system. The implantable unit delivers stimulation with characteristics (e.g., intensity, pulse width, duration, etc.) based on a selected stimulation procedure. The implantable unit receives sensor information from one or more implanted sensors in the subject (e.g., sensors implanted along a surface of the subject's brain). The implantable unit includes a classifier, which selects a stimulation procedure based on the sensor information. The classifier may indicate a clinically relevant state of the subject. For example, in an embodiment where the implantable unit is to manage ET, the implantable unit may have two stimulation procedures ‘high’ and ‘low’ and if the classifier indicates that the patient is active, then the implantable unit may provide the ‘high’ stimulation procedure and may provide the ‘low’ stimulation procedure otherwise. The classifier may be trained based on an external unit which is not implanted in the subject. For example, the classifier may be trained using a machine learning model.

In an example training procedure, the external unit may receive the sensor information from the implanted sensors. The sensor data may be classified based on symptom information. For example, the symptom information may be obtained using one or more additional sensors (which may be implanted or externally attached to the subject), the implanted sensors, manually input by a clinician and/or subject, registered based on time stamped information in the sensor information, or combinations thereof. The symptom information may label periods of the sensor data. A machine learning model is trained to generate a classifier based on the classified sensor data. The classifier may be loaded onto the implantable unit. In this manner, a classifier may be trained to determine clinically relevant states of the subject based on the sensor information from the implantable sensors. The use of an external device may allow for increased flexibility and processing power during the training of the classifier. For example, during training, a bias may be applied to the classifier to alter the diagnostic characteristics of the classifier (e.g., to reduce the false negative rate). Similarly, the implantable unit may have relatively limited processing power. The use of an external unit may allow for a more sophisticated classifier to be trained than would be possible with the implantable unit alone.

In addition, the use of training the classifier based on symptom information may provide a benefit by allowing the classifier to associate the implantable sensor information with markers of the subject's condition. This may allow the implantable unit to operate based off the implantable sensors, which in turn may reduce or eliminate the need for the subject to wear additional devices during the normal operation of the implantable unit. For example, the classifier may determine if the subject is active or at rest based on the implantable sensors, without the need for accelerometer data. Once the classifier is trained and loaded into the implantable unit, the implantable unit may be able to operate without the need for connection to external sensors or systems.

While the present disclosure is generally described with respect to an example embodiment where the system is an aDBS system used to treat ET, it should be understood that the present disclosure is not limited to either aDBS systems or to their use to treat ET. For example, in some embodiments, the present disclosure may relate to a stimulation system which is used to treat epilepsy, pain, psychiatric disease (e.g., obsessive-compulsive disorder, Tourettes, depression), obesity, addiction, self-injurious behavior, ET, Parkinson's disease, movement disorders, or combinations thereof.

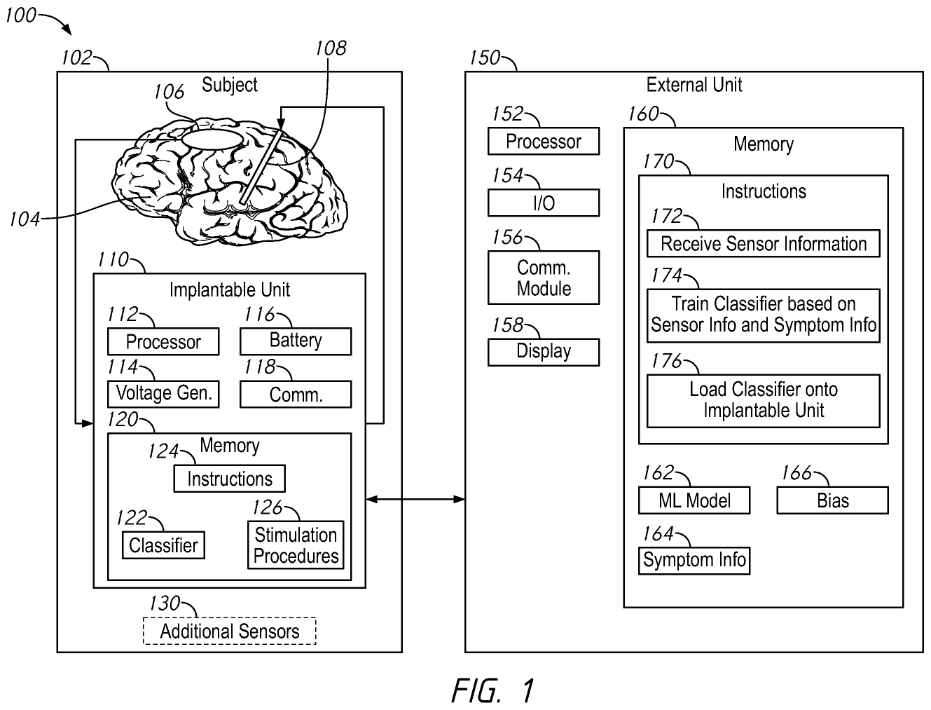

FIG. 1 is a block diagram of a distributed training system for an implantable device according to some embodiments of the present disclosure. The distributed training system 100 includes an implantable unit 110 which includes one or more components implanted in a subject 102 and an external unit 150 which includes one or more components which are not implanted in the subject 102. The implantable unit 110 may manage stimulation of the nervous system 104 of the subject 102, such as the subject's brain. The implantable unit 110 may provide electrical signals to a stimulation electrode 108 implanted in the nervous system 104. The implantable unit 110 may also be coupled to implanted sensors 106 which may measure one or more aspects of the subject 102. The implantable unit 110 uses a classifier 122 to classify a state of the subject 102 based on information from the implantable sensors 106. Based on the classification, the implantable unit 110 may select a stimulation procedure 122. During a training procedure, the implantable unit may be communicatively coupled to the external unit 150, which may generate the classifier 122 based on information from the sensors 106. The classifier may then be loaded onto the implantable unit 110 and the external unit may be uncoupled to allow the implantable unit to function without the need for external connections.

The implantable unit 110, sensors 106 and electrode 108 may be part of an implantable system which is implanted in a subject 102. The subject 102 may be a patient who suffers from one or more neuromuscular conditions. For example, the subject 102 may be a human being who has a movement disorder such as essential tremor (ET) and the implantable system may be an aDBS system. The present disclosure is not limited to any, particular condition, type of subject, or type of stimulation. For example, in some embodiments, the subject may be a non-human animal (e.g., for veterinary applications) and/or may be a subject who is implanted for research purposes without necessarily having a diagnosed condition.

The subject 102 may be implanted with an implantable system including a stimulation electrode 108, one or more implantable sensors 106 and an implantable unit 110. The implantable unit 110 (e.g., an IPG) may apply electrical signals to the stimulation electrode 108 based, at least in part, on signals the implantable unit 110 receives from the implantable sensors 106. As an example application, the features of FIG. 1 may generally, be described with respect to an adaptive deep brain stimulation (aDBS) system, where the electrode 108 is implanted in the subject's 110 brain 104, and the sensors 106 are electrocorticography (ECoG) sensors implanted along a surface of the brain 104 to measure a local field potential (LFP). However, other example applications may include other locations for the electrode 108 and/or location/type of sensor.

The implantable unit 110 may operate the electrode 108 based on sensor information from the sensors 106 as well as a classifier 122 loaded in a memory 120 of the implantable unit 110. In some embodiments, the sensor information may also include information from the electrode 108. In some embodiments, the implantable unit 110 may be a controller such as an IPG which is implanted in a location distal from the sensors 106 and electrode 108. For example, the sensors 106 and electrode 108 may be implanted on, in, or near a brain 104 of the subject 102, while the implantable unit 110 may be implanted in another location, such as in a chest cavity of the subject 102. The implantable unit 110 may be coupled to the sensors 106 and/or electrode 108, for example with wires which are implanted in the subject 102. The implantable unit 110 may be a self-contained unit, or may include one or more modules coupled together (e.g., a first unit with the processor 112 and memory 120 coupled to a second unit with the battery 116).

The implantable unit 110 includes a processor 112 which operates the implantable unit 110 based on information loaded in a memory 120 of the implantable unit 110. The implantable unit 110 may also include a battery 116, which may power the implantable unit 110 as well as provide power for driving signals to the electrode 108 which provide the stimulation to the nervous system 104 of the subject 102. In some embodiments, the battery 116 may also provide power to the sensors 106, in some embodiments, the sensors 106 may be passive and may not require external power. The implantable unit 110 also includes a voltage generator 114 which the processor 112 may operate to apply electrical signals to the electrode 108 as part of a selected stimulation procedure 126. The implantable unit 110 also includes a communication module 118 which may be used to communicatively couple the implantable unit 110 to send and receive information from external devices, such as the external unit 150.

The implantable unit 110 also includes a memory 120, which includes instructions 124 as well as other information the processor 112 may use to operate the implantable unit 110. For example, the memory 120 may include multiple stimulation procedures 126, which include information about how the processor 112 should operate the voltage generator 114 to apply stimulation via the electrode 108. For example, each stimulation procedure 126 may specify one or more waveforms to be used to generate stimulation signals. In some embodiments, the implantable unit 110 may apply a pulsed signal to the electrode 108, and the stimulation procedure may specify properties such as duration, amplitude, duty cycle, pulse width, number of pulses, frequency, etc. The stimulation procedure may also indicate which electrodes to activate in embodiments where the stimulation electrode 108 includes more than one electrode.

The memory 120 of the implantable unit 110 may include multiple stimulation procedures 126. For example, the memory 120 may include a ‘high’ stimulation procedure and a low′ stimulation procedure. For example, the ‘low’ stimulation procedure may have a lower voltage intensity than the ‘high’ stimulation procedure. In some embodiments, the ‘low’ procedure may set a voltage intensity to 0V, and no stimulation may be applied while the low procedure is in use. Other numbers and types of stimulation procedures may be used in other example embodiments.

In some embodiments, the different stimulation procedures may be determined by a clinician. For example, during a set up after implantation of the electrode 108 and implantable unit 110, a clinician may modify various settings. In some embodiments, the clinician may modify (and/or use ‘as-is’) one or more parameters of a pre-set program. For example, the clinician may begin with a ‘default’ program, observe its effect on the subject, and modify one or more parameters, and repeat the process. In some embodiments, the stimulation procedures may have parameters which are developed, at least in part, automatically. For example, a machine learning model may be trained with different patient responses and the trained machine learning model may generate a set of parameters for a stimulation procedure. In some embodiments, the machine learning generated stimulation procedure may be further adjusted by a clinician. In some embodiments, the stimulation procedures 126 may be trained on the same external unit 150 used to generate the classifier 122. In other embodiments, the training of the classifier 122 and stimulation procedures 126 may be performed separately.

The memory 120 of the implantable unit 110 includes a classifier 122 which when operated by the processor 112 interprets information from the implantable sensors 106 (and/or electrode 108) to determine a state of the subject 102. Based on the determined state of the subject 102, the processor 112 may select one or more of the stimulation procedures 126. For example, if the subject 102 is in a first state, a first stimulation procedure may be selected and performed, if the subject 102 is in a second state, a second stimulation procedure may be selected and performed, and so forth. In some embodiments, the classifier 122 may be a binary classifier which selects between two possible states. In an example embodiment where the subject 102 is an ET patient, the classifier 122 may be a binary linear classifier which uses the sensor information to determine if the subject 102 is active or is at rest. If the classifier 122 determines the subject is active, then a ‘high’ stimulation procedure may be selected, while if the subject is at rest, then a ‘low’ stimulation procedure may be used (since tremor may not be as pronounced while the subject is at rest).

In some embodiments, instead of (or in addition to) determining an ongoing state of the subject 102, the classifier 122 may detect events in the subject's nervous system 104. For example, the classifier 122 may detect neurological markers of one or more neurological events for logging or diagnostic purposes.

The implantable unit HO may function in this manner as a self-contained system, classifying sensor information from the sensors 106 and using that classified sensor information to determine which stimulation procedure 126 to perform via the electrode 108. During a normal operation of the implantable system, no external connections (e.g., to devices on or outside the subject 102) may be needed. For example, the implantable unit 110 may classify a state of the subject 102 based on the sensor information from the implantable sensors 106 (and/or electrode 108), without the need for any additional sensors (e.g., an IMU worn on the subject 102). The implantable unit 110 may still couple to outside devices for updates, to provide feedback and/or for diagnostic/testing purposes. The implantable unit 110 may also couple to the external unit 150 in order for the classifier 122 to be trained and loaded onto the implantable unit 110.

The classifier 122 may be generated based on a distributed training system 100 which includes an external unit 150. The external unit 150 may include one or more computing systems which can be communicatively coupled to the implantable unit 110. For example, the external unit 150 may include a general purpose computing device such as desktop computer, a laptop, a tablet, a smartphone, and so forth. In some embodiments, the external unit 150 may include multiple devices. For example, a mobile device such as a smartphone or tablet may act as an interface between the implantable unit 110 and one or more additional computers (e.g., a desktop, one or more networked devices). In some embodiments, one or more of the functions of the external unit 150 may reside in networked devices (e.g., in a cloud computing system).

The external unit 150 includes a processor 152 which executes various instructions in a memory 160 to generate a classifier, an input/output system 154 which allows a user to interact with the external unit 150, a communications module 156 which allows communication between the external unit 150 and the implantable unit 110 (along with other external devices), and a display 158 which allows a user to visualize information about the external unit 150 and/or implantable unit 110.

The external unit 150 includes a memory 160, which includes instructions 170 for training a classifier which may be loaded onto the implantable unit 110, as well as other information which may be useful for training the classifier, such as a machine learning (ML) model 162, symptom information 164, and bias information 166.

The external unit 150 may be communicatively coupled to the implantable unit 110 during a training process. For example, during the training process, a communications module 156 of the external unit 150 may be in communication with a communications module 118 of the implantable unit 110. The external unit 150 and implantable unit 110 may be coupled via wired connections, wireless connections, or combinations thereof. For example, the external unit 150 may be coupled to the implantable unit 110 via Bluetooth, or some other communications protocol. In some embodiments, a device specific connection protocol (e.g., a proprietary wireless communications protocol) may be used. In some embodiments, a proprietary system may act as an intermediary between the implantable unit and a more general external system. For example, the implantable unit 110 may communicate with a proprietary system such as a proprietary tablet via a device specific connection protocol and the proprietary system may communicate with a more generalized external system over a more generalized connection protocol (e.g., wi-fi Bluetooth, etc.)

The external unit 150 includes instructions 170 which may be executed by the processor 152 to as part of the training process to train a classifier. For example, the instructions 170 may include a step 172 for receiving sensor information from the implantable unit. A step 174 for training a classifier based on the sensor information as well as symptom information 164, and a step 176 for loading the classifier onto the implantable unit 110. Once the classifier is loaded onto the implantable unit 110 (e.g., as classifier 122), the external unit 150 may be decoupled from the implantable unit and the implantable unit may operate on its own.

The step 172 describes receiving sensor information from the implanted sensors 106.

For example, the communications module 118 of the implantable unit 110 may provide information it is receiving from the sensors 106 (and/or electrode 108). In some embodiments, the sensor information may be provided ‘live’ as it is being received by the implantable unit (or as quickly as the implantable unit 110 is able to process and provide such information). In some embodiments, the sensor information may be recorded by the implantable unit 110 and provided in pre-recorded segments. The sensor information may include raw information from the sensors 106, processed information that the implantable unit derives from that raw information or combinations thereof. For example, the implantable unit 110 may measure a bandpower of the sensor information and provide the bandpower to the external unit. In some embodiments, the sensor information may include information from the stimulation electrode 108. For example, the implantable unit 110 may record information from electrodes 108 not currently used for stimulation, or from electrodes 108 in-between stimulation pulses.

The symptom information 164 may indicate a status of the subject 102 which can be correlated to the sensor information. For example, the symptom information 164 may mark whether a certain portion of the sensor information was recorded while the subject 102 was active. The symptom information 164 may be generated manually, automatically, or combinations thereof. For example, a clinician may monitor the subject 102 and manually annotate the sensor information with symptom information (e.g., by marking a section of sensor data based on an observed state of the subject). In another example, the subject 102 may be directed to perform certain actions at certain times (e.g., be active during a first time period and be at rest during a second time period) and the time information during which they were performing these tasks may be used to correlate symptom information to the sensor information.

In some embodiments, one or more additional sensors 130 may measure symptom information of the subject 102. For example, if the desired symptom information is related to the motion of the subject 102, then one or more accelerometers may be attached to the subject 102 during the training process which may record information about the subject's movement. Based on the information from the additional sensors 130, the symptom information 164 may be generated. In some embodiments, the additional sensors 130 may only be present during a training process, and may not be otherwise present (e.g., the additional sensors 130 may be removed after a training process is complete).

The symptom information 164 may take the form of labels applied to different periods of the sensor information used as training data. For example, sensor information may be recorded during a first period and labelled as a first subject state based on the symptom information 164, and recorded during a second period and labelled as second subject state based on the symptom information 164. For example, the subject may be directed to be at rest during the first period and active during the second period, and the known activity of the subject may be used as the symptom information 164 to label the sensor information used as training data. The sensor information may include time-stamped data, which may be matched to known periods where the subject was active or at rest.

The external unit 150 may use a machine learning (ML) model 162 to generate the classifier. For example, the machine learning model 164 may be trained based on the sensor information from the sensors 106 and the symptom information 164 to determine a state of the subject 102 based on the sensor information. For example, the symptom information 164 may represent the state of the subject (or markers associated with that state) that it is desired for the classifier 122 to detect (e.g., an active state or an at rest state). The ML model 162 may develop a classifier which detects the states based on the information from the sensors 106 which is available to the implantable unit 110. In some embodiments, the classifier 122 may be trained on a patient by patient basis (e.g., due to inter-patient variability). The ML model 162 may be trained via unsupervised learning, supervised learning, or combinations thereof.

In some embodiments, a bias 166 may be applied to the classifier. For example, in an embodiment where the classifier 122 determines between two states, it may be desirable to preferentially bias the classifier to one state over the other. In an example embodiment where the classifier 122 selects between an active and an at rest state, it may be useful to have the classifier 122 select the ‘active’ state more often than the at rest state, since the active state may cause stimulation to be provided, and a false positive is more desirable than a false negative (where no stimulation is provided even though stimulation is needed). The bias 166 may be a user selectable and/or adjustable feature. For example, a user may ‘tune’ a level of the bias 166. The user may set a desired amount of bias 166 and the ML model 162 may be trained taking the bias 166 into account such that the classifier is generated with the bias 166 built-in.

In some embodiments, a user of the system 100 may provide feedback or input which guides the training of the classifier. For example, the subject may use the I/O 154 to provide patient feedback which may be indicative of potential side-effects, impacts of stimulation, classifier performance, or combinations thereof. During the training, the external unit 150 may display information (e.g., on display 158) which users may allow users to monitor the training process. For example, the display 158 may show a visualization of the training data and/or a visualization of the classifier operating on the training data (e.g., a visualization of a threshold set by the classifier). In some embodiments, the user's input may be guided by observing the information during the training.

In some embodiments, step 174 may be iteratively repeated. For example, a classifier may be trained, evaluated, and iteratively improved. For example, the step 174 may include iteratively repeating classifier training using performance results of previous iterations to further enhance performance. In some embodiments, a user may be presented with options between iterations to provide input and/or feedback to guide subsequent iterations.

The instructions 170 include step 176, which describes loading the trained classifier onto the implantable unit. For example, the trained classifier may be loaded into the memory 120 as classifier 122. In some embodiments, once the classifier is loaded onto the implantable unit 110 communications may cease between the implantable unit 110 and the external unit 150. In some embodiments, the subject 102 may periodically undergo the training process again to update the classifier on their implantable unit 110.

The external unit 150 may include an input/output (I/O) system which allows a user to interface with the external unit 150 (and through it, potentially with the implantable unit 110). For example the I/O system 154 may include a mouse, keyboard, touchscreen, voice control, or combinations thereof. The external unit 150 may include a display, such as a monitor, screen, printer, or combinations thereof which allow information to be displayed to a user.

In some embodiments, the features of the external unit 150 may be distributed across one or more devices, some of which may be remote from the subject 102. For example, the external unit 150 may include one or more networked devices (e.g., as part of a cloud computing system) which interact with the implantable unit 110. In an example embodiment, device proximal to the subject 102, such as a tablet, may communicate with the implantable unit 110, and then communicate across a network (e.g., via the internet) with one or more computing units in remote locations, which may perform the instructions 170.

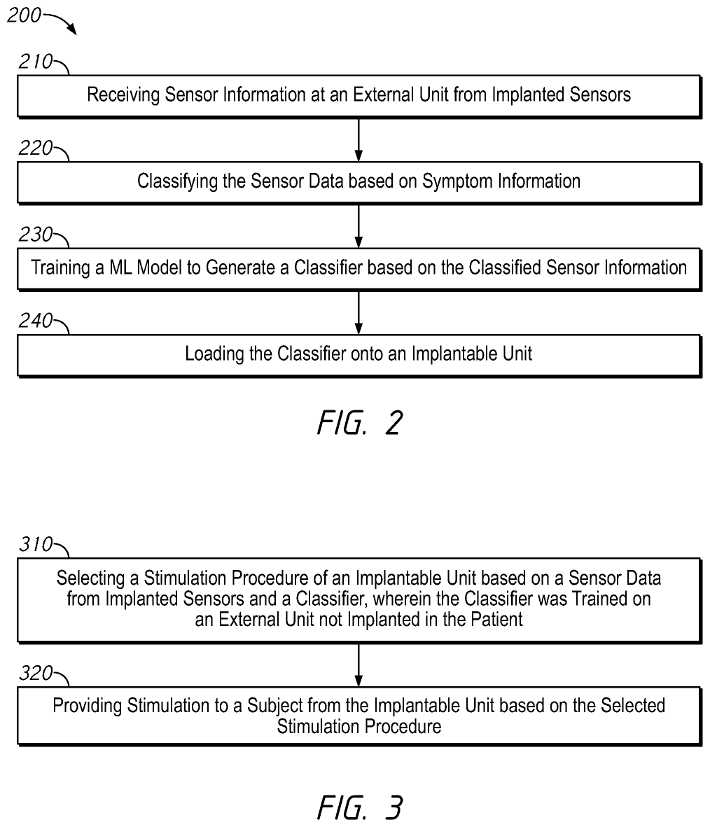

FIG. 2 is a flow chart of a method of training a classifier for an implantable system according to some embodiments of the present disclosure. The method 200 may, in some embodiments, be performed by the training system 100 of FIG. 1, For example, blocks 210 to 240 of FIG. 2 may be implemented by the instructions 170 of FIG. 1. The steps of method 200 may generally be performed by an external unit (e.g., 150 of FIG. 1) which is communicatively coupled to an implantable system (e.g., which includes implantable unit 110, sensors 106, and electrode 108 of FIG. 1). The method 200 represents a training procedure which may be performed to generate and load a classifier onto the implantable system.

In some embodiments, the method 200 may begin with communicatively coupling the external unit to the implantable system. For example, the method 200 may include establishing a wired or wireless connection between a communications module of the external unit and a communications module of the implantable system. In some embodiments, one or more components of the external unit may be remote from the subject with the implantable system. For example, the implantable system may communicate with a local device (e.g., a tablet, a computer, smartphone) which may then communicate with remote components of the external system (e.g., in the cloud over the internet).

The method 200 includes block 210, which describes receiving sensor information at an external unit from implanted sensors. For example, the method 200 may include communicatively coupling an external unit (e.g., 150 of FIG. 1) to an implantable unit (e.g., 110 of FIG. 1) of a subject and receiving the sensor information from the implantable unit. In some embodiments, the method 200 may include generating the sensor information with the implantable unit based on raw sensor data. The sensor information may be received ‘live’ (e.g., the information may be streamed at the time or soon after being collected) or pre-recorded segments of information may be received. In some embodiments, the sensor information may include information from stimulation electrodes (e.g., 108 of FIG. 1), from implantable sensors not used for stimulation (e.g., 106 of FIG. 1), or combinations thereof.

Block 220 generally follows block 210 and describes classifying the sensor information based on symptom information. For example, the symptom information may include information about a condition of the subject and/or information which is a proxy for information about a condition of the subject. The classifying of block 220 may include labelling periods of the sensor information based on the symptom information. For example, labelling a first period of the sensor information as an ‘at rest’ period and a second period as an ‘active’ period. In some embodiments, the classification of the sensor data may be manual, automatic, or combinations thereof. In some embodiments, the method 200 may include classifying the sensor information based, at least in part, on a clinician's judgement. In some embodiments, the method 200 may include collecting information from an additional sensor (e.g., an additional sensor placed on the patient) and classifying the sensor data based, at least in part, on data from the additional sensor.

Block 220 may generally be followed by block 230, which describes training a machine learning model to generate a classifier based on the classified sensor information. For example, the machine learning model (e.g., 162 of FIG. 1) may use the classified sensor information to determine the characteristics of the sensor information available to the implantable unit which indicate the symptom information. For example, the classifier may be a binary classifier, such as a binary linear classifier, which distinguishes between a first state of the subject and a second state of the subject. For example, the sensor information may be classified with symptom information which indicates whether the sensor information was collected while the subject was active or while the subject was at rest. The classifier is trained to determine if the subject is active or at rest based on the sensor information.

In some embodiments, the method 200 may include asking the subject to perform one or more tasks to gather sensor information under different conditions. For example, the method 200 may include collecting a first set of sensor data while the subject is at rest and a second set of sensor data while the subject is performing a task. The sensor information may be time-stamped, and the method 200 may include correlating the time-stamped sensor information with periods of known activity of the subject.

In some embodiments, the method 200 may include applying a bias to the classifier. For example, if the classifier is a binary-classifier, it may be desirable to bias the classifier to preferentially select one outcome (e.g., to reduce the number of false negatives). The method 200 may include selecting a bias and adjusting the classifier based on the bias. For example, the method 200 may include training a machine learning model to generate the classifier based, in part, on the bias.

In some embodiments, the method 200 may include iteratively repeating the training of the ML model to generate the classifier. For example, the method 200 may include iteratively repeating classifier training using performance results of previous iterations to further enhance performance.

In some embodiments, the method 200 may include providing feedback or input which guides the training of the ML model. For example, the subject may provide patient feedback which may be indicative of potential side-effects, impacts of stimulation, classifier performance, or combinations thereof. In another example, a clinician may select a level of bias to apply to the classifier.

Block 230 may generally be followed by block 240, which describes loading the classifier onto an implantable unit. For example, the classifier trained by the external unit may be loaded onto a memory (e.g., 120 of FIG. 1) of the implantable unit. In some embodiments, after loading the classifier, the method 200 may include decoupling the external unit from the implantable unit (e.g., ending wireless communication).

FIG. 3 is a flow chart of a method of providing stimulation with an implantable system according to some embodiments of the present disclosure. The method 300 may represent steps which are performed by an implantable system (e.g., implantable unit 110, sensors 106, and electrode 108 of FIG. 1). The method 300 may represent a ‘normal’ operational state of the implantable system after a training procedure (e.g., after the steps of method 200 of FIG. 2), The method 300 may follow the method 200 of FIG. 2, however, the steps of method 300 may be performed without an external system (e.g., 150 of FIG. 1) being coupled to the implantable system.

Blocks 210 to 240 of FIG. 2 may generally describe a training process for a classifier of an implantable system. Blocks 310 and 320 of FIG. 3 may describe the operation of the implantable system once the trained classifier is loaded. Blocks 310 and 320 may be performed without the external system being coupled to the implantable system. For example, in some embodiments, steps 210 to 240 may be performed while the subject is at a check-up or other clinical setting, and blocks 310 and 320 may represent steps performed during the normal day to day life of a subject.

Block 310 describes selecting a stimulation procedure of the implantable unit based on the sensor data from the implanted sensors and the classifier. The classifier may be trained on an external unit which is not implanted in the patient. For example, the classifier may be trained using the method 200 of FIG. 2. In some embodiments, the classifier may be trained during an initialization of the implantable unit. In some embodiments, the classifier may be re-trained as part of an update process. For example, the classifier may be re-trained as part of a regular check-up process.

In some embodiments, the classifier may be a binary classifier with a first result and a second result, the implantable unit may have a first stimulation procedure associated with the first result and a second stimulation procedure associated with the second result. If the classifier indicates the first result based on the current sensor information, then the first stimulation procedure may be selected, if the classifier indicates the second state, then the second stimulation procedure may be selected.

Block 310 may generally be followed by block 320, which describes providing stimulation to the subject from the implantable unit based on the selected stimulation procedure. For example the stimulation procedure may indicates stimulation parameters such as a voltage intensity, pulse width, pulse duration etc. The method 260 may include applying a stimulation signal to one or more implanted electrode (e.g., 108 of FIG. 1) based on the selected stimulation procedure. In some embodiments, the method 300 may include using the stimulation to treat epilepsy, pain, psychiatric disease (e.g., obsessive-compulsive disorder, Tourettes, depression), obesity, addiction, self-injurious behavior, ET, Parkinson's disease, movement disorders, or combinations thereof.

### Example

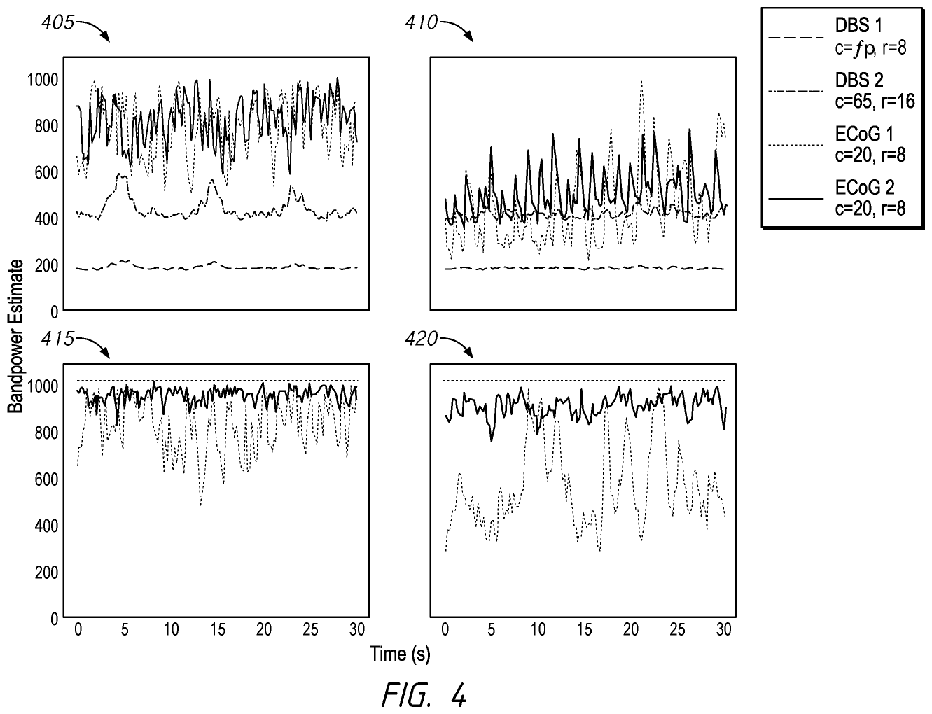

FIGS. 4-6 show example data from an example implementation where a binary classifier is trained on an external unit and used to manage an adaptive deep brain stimulation (aDBS) system based on signals from an ECoG-sensing strip. The examples of FIGS. 4-6 shows an example embodiment and the present disclosure is not limited to the described details. For example, the present disclosure is not necessarily limited to aDBS systems, ECoG sensors, or binary classifiers, or any of the other details described in this example. The Example of FIGS. 4-6 also describes example results and procedures with respect to two different subjects, patient 1 (P1) and patient 2 (P2) to illustrate inter-subject variability. It should be appreciated that certain values given herein may be specific to the described examples and that embodiments may have different classifiers, stimulation procedures, amplitudes of stimulation, etc.

Subject Information and Device Specifications

In some examples, two subjects diagnosed with ET were implanted with an IPG, specifically an Activa PC+S (e.g., implantable unit 110 of FIG. 1), a neurostimulator with a DBS lead (e.g., a DPBS probe such as electrode 108 of FIG. 1) implanted unilaterally in the ventral intermediate nucleus (VIM) thalamus and an ECoG-sensing strip of electrodes (e.g., sensor 106 of FIG. 1) placed over the hand portion of the ipsilateral motor cortex. This system is capable of recording neural data in the form of local field potential (LFP) data from each component of the device and either streaming the raw data directly to an experimental computer (e.g., external unit 150 of FIG. 1), or of computing an on-board estimate of the bandpower of a given band of this raw neural data which may, itself, be either streamed or used for on-board processing. As should be appreciated, while a specific implantable unit, an Activa neurostimulator, is described throughout, other neurostimulators may be used as the implantable unit, and discussion of the Activa PC+S neurostimulator is in no way limiting. Similarly, other types of sensors beyond electrode sensing strips may be used, and use of an ECoG-sensing strip of electrodes is in no way limiting.

The on-board classifier of the implantable unit discussed in this example uses a bandpower estimate of a given band of LFP data, calculated on-board the implantable unit, to classify data. The band may be of the form c±r with c={2.5, 5, 7.5 . . . 97.5, 100}Hz and r={2.5, 8, 16}Hz, with an estimate generated, for example, every 200 ms (5 Hz). In some examples, at least two different bandwidths may be collected from the DBS probe and the ECoG strip, respectively, resulting in a state estimate \hat{X} ∈R^{4×1} defined by equation 1, below:

$$\hat{X} = [ \begin{matrix}{x}_{DBS , 1} \\ {x}_{DBS , 2} \\ {x}_{ECoG , 1} \\ {x}_{ECoG , 2}\end{matrix} ] \qquad Eqn . 1$$

The state estimate \hat{X} is matrix which includes the band power χ for the DBS electrode in a first and second bandwidth χ_{DBS,1} and χ_{DBS,2} as well as band power for the ECoG in the first and the second bandwidth χ_{ECog,1} and χ_{ECog,2}. The state estimate \hat{X} may be used by the implantable unit to determine a stimulation procedure (e.g., 126 of FIG. 1) to provide.

For example, a binary linear classifier (e.g., 122 of FIG. 1) may be used to select a stimulation procedure (e.g., 126 of FIG. 1) based on the state estimate \hat{X}. For example, the results of the classifier may be used to select between a stimulation procedure with an amplitude set to a pre-determined “LOW” value (set in this example to 0.0 V in both subjects) or a stimulation procedure with an amplitude set to a “HIGH” value (e.g., a clinician-determined, patient-specific amplitude). In this example embodiment, other parameters (frequency, pulse width, stimulation electrode configuration) of the stimulation procedure may be held constant between the two procedures. Other embodiments may vary other parameters beyond amplitude between stimulation procedures 126.

Distributed Training Architecture

During a training of the classifier (e.g., as part of the training system 100 of FIG. 1 or during the method 200 of FIG. 2), training data may be directly streamed from the implantable unit such as the IPG (or other suitable devices described herein) to an external unit, such as an experimental computer (or other computing device). This may allow for accurate and reliable time-stamped data collection, and may permit the instantaneous review of all training data to easily determine if a test should be repeated. In some cases, when results have been obtained, the classifier may be uploaded to the IPG itself for evaluation of aDBS in free movement.

FIG. 4 shows a set of graphs which represent example training data according to some embodiments of the present disclosure. The training data represented by the graphs 405 to 420 may, in some embodiments, but used to train a classifier (e.g., as part of the step 174 of FIG. 1 and/or the method 200 of FIG. 2). The graphs 405 to 420 show a state estimate \hat{X} generated by the implantable unit (e.g., 110 of/FIG. 1) based on the implantable sensors (e.g., 106 of FIG. 1) and from a stimulation procedure applied to an electrode (e.g., 108 of FIG. 1) over time. As described in Eqn. 1, above, the state estimate \hat{X} includes multiple values which are shown as different traces on each of the graphs.

In the example embodiment of FIG. 4, the symptom information used to train the classifier (along with the training data in the graphs 405 to 420) may be based on the state of the subject and the stimulation used to collect each set of data. Because the presence of stimulation is known to alter neural dynamics, data may be collected in each state with stimulation both active and disabled. Each of the graphs 405 to 420 shows 30 seconds of data during different conditions of the stimulation procedure and the activity of the subject. Graph 405 shows no stimulation while the subject is at rest, graph 410 shows no stimulation while the subject is active, graph 415 shows stimulation while the subject is at rest, and graph 420 shows stimulation while the subject is active.

A machine learning model (e.g., 162 of FIG. 1) may be provided the training data along with the labels of the subject state and stimulation state (e.g., [active, off] for graph 410). The machine learning model may train on this information to generate a classifier which determines between the different subject states (e.g., active or at rest).

Supervised Training Data Collection

As an example procedure to obtain data during each of the four possible patient states described above, in one example, 30 seconds of data were collected with the patient at rest with hands in lap with stimulation active and with stimulation disabled, and 30 seconds while the patient was continuously conducting the finger-to-nose task of the Fahn-Tolosa-Marin (FTM) tremor rating scale with stimulation active and with stimulation disabled. The example data shown in graphs 405-420 (e.g., two minutes' total data) may be used to train an intrinsically personalized classifier, such as the classifier 122 of FIG. 1.

Following data collection, a visualization of the time series of the bandpower estimate data similar to the graphs of FIG. 4, along with cross-validated accuracy of a classifier trained on this data, was available for immediate review by a user (e.g., a clinician, researcher and/or the subject). For example, the visualization may be presented on a display (e.g., 158 of FIG. 1) for user review. This may inform the user's decision to repeat individual tests if necessary. The structure of the training data collection process was arranged to allow for individual states to be recorded independently, as opposed to necessitating a complete repetition of the full training procedure.

As may be seen in the graphs of FIG. 4, differences between the Rest and Action states are apparent for ECoG data within stimulation states, with clear differences between the behavior across stimulation states. In the DBS channels, note the differences between signal behavior during Rest and Action with stimulation Off. During stimulation On in graphs 415 and 420, the configured bandpower estimates for both DBS channels are saturated; this is indicated by the solid lines at the maximum value the system may output. This implies that the DBS channels provide both an effective indication of whether stimulation is active and useful information when it is not.

Band Selection and Algorithm Design

In some embodiments, the implantable unit may differentiate between when stimulation may be set to “HIGH”, and when it may be left “LOW” based on the results of the classifier. It may be useful, therefore, for the classifier to be trained to determine when a patient requires stimulation to treat their symptoms, which in ET patients may be said to be the difference between when the patient is at Rest and generally without tremor, and when they are in Action and thus experiencing tremor.

Additionally, in some examples, the classifier may be unable to “know” whether stimulation is active or disabled. Accordingly, in such examples, some method of indirect inference for device state may be provided. In some examples, to allow the implantable unit to determine whether stimulation was active or disabled, χ_{DBS,1} bandpower estimate was set to the patient-specific, clinically determined stimulation frequency, (c=fₚₐₜᵢₑₙₜ, r=8). χ_{DBS,2} bandpower estimate was set to measure thalamic γ-band (c=65, r=16), previously demonstrated to correlate with movement. Although suppression of thalamocortical coupling between the γ-band of the cortex and lower frequency bands of the VIM have been demonstrated to strongly correlate with movement, the noise floor in the Activa PC+S ECoG strip precludes effective measurement of this range. Instead, both χ_{ECoG,1} and χ_{ECoG,2} were set to record β-bandpower (c=20, r=8) from alternating pairs of the 4 linearly arranged ECoG electrodes available, desynchronization of which is known to correlate with movement onset, thus indirectly indicating onset of tremor in ET. As should be appreciated, additional and/or alternative c and r values may be implemented, and are considered to be within the scope of this disclosure.

With \hat{X} thus defined, an unbiased linear classifier was trained (e.g., via the process of method 200 of FIG. 2) to differentiate between the 60 seconds of data collected during the Rest recordings and the 60 seconds collected during the Action recordings. A linear projection ŷ₀∈R was defined with ω ∈R^{1×4}, b∈R, norm₁∈R^{4×1} and norm₂∈R^{4×1} of the form of Equation 2, below:

ŷ₀=−ω(\hat{X}−norm₁)⊙norm₂)+b Eqn. 2

so as to maximize the variance between of ŷ_{0,Rest} and ŷ_{0,Action}. In an example unbiased classifier, a sample with a value above 0 was classified as Action and a value below 0 as Rest.

Under the clinically informed theory that it is preferable to have stimulation unnecessarily active than to risk it being absent when needed, in some embodiments the classifier may be biased in favor of keeping stimulation On. For example, a bias (e.g., 166 of FIG. 1) may be selected. In some embodiments, the bias may be user selectable. In some embodiments, the bias may be expressed as a number of standard deviations to move a threshold. For example, the bias may be set at λ=⅕ the standard deviation of the projection from training data. In such embodiments, this may be accomplished by adding λ to that projection, thus creating the final projection ŷ=ŷ₀+λ. This fraction may be determined through analysis of previously recorded data for the aDBS systems and found to reduce false negatives without excessively increasing the overall error rate. It may be advantageous to take an unbiased classifier and, using the statistical characteristics of the training data recorded to generate this classifier, bias it in favor of maintaining active stimulation.

Although in this case, the bimodal distribution of the data led to the decision to use

$$\frac{1}{5}$$

of the standard deviation of the data, as should be appreciated, this fraction may be adjusted as needed depending on the statistical characteristics of the relevant training data.

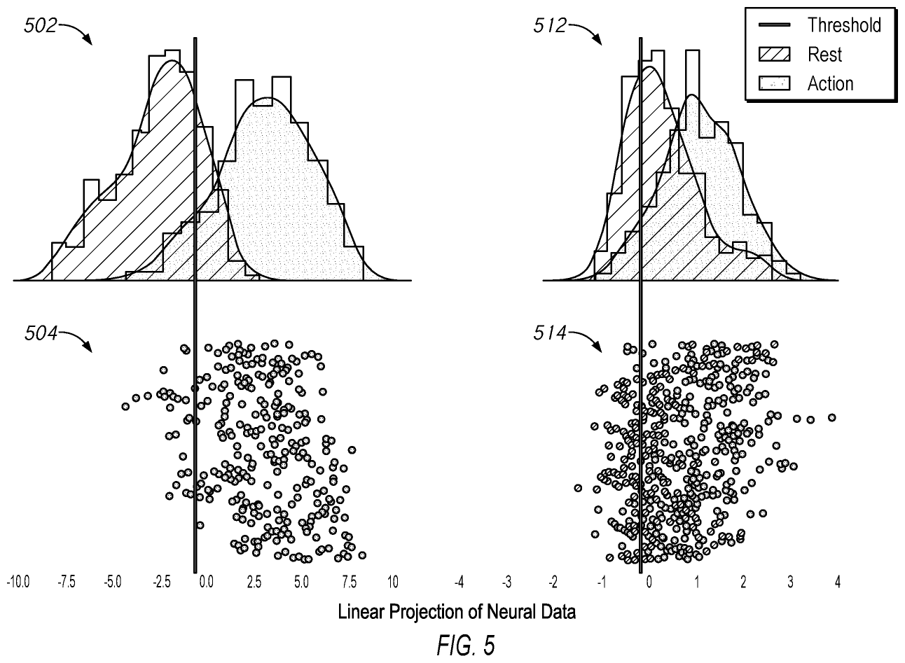

FIG. 5 shows graphs of example distributions of measured data and biased classification thresholds according to some embodiments of the present disclosure. The graphs 502 to 514 represent visualizations of data categorized by a classifier (e.g., 122 of FIG. 1). The graphs 502 to 514 may represent measurements based on the training data represented in FIG. 4, The graphs 502 and 504 represent sensor information from a first subject P1 and the graphs 512 and 514 represent sensor information from a second subject P2.

The classifier may set a threshold based on the training data (shown as the vertical line). In the graphs 502 to 514, the horizontal axis has been normalized such that in an unbiased classifier, any value below zero would return a first result (e.g., at rest) and any value above zero would return a second result (e.g., active). In the example embodiment of FIG. 5, as described above, the classifier has been biased to reduce false negatives by shifting the threshold below zero (e.g., to the left as shown on the graphs 502 to 514). Accordingly, any value below the threshold may return the first result and any value above the threshold may return the second result.

Performance of Biased Classifier on Training Data

For the training data, a false positive is said to have occurred if a sample labelled Rest is above the threshold, while a false negative is said to have occurred if a sample labelled Action is below the threshold. Total error rate is the average of these rates, as equal training time was spent in the Rest and Action states. In this example, biasing the threshold resulted in a 12.7% increase in average error rate; however, this constituted a 28.2% decrease in average false negative rate as shown in Table 1, below. Though overall error rate is increased by a marginal amount, clinically relevant false negative rate may be reduced in both patients, indicating that biasing was an effective method for increasing treatment reliability by clinical considerations.

TABLE 1
Effects of biasing on training data

| Measure | Unbiased | Biased |
|---|---|---|
| P1 training error rate | 0.122 | 0.143 |
| P2 training error rate | 0.342 | 0.380 |
| P1 training false neg. rate | 0.130 | 0.090 |
| P2 training false neg. rate | 0.097 | 0.073 |

Tremor Severity Characterization and Analysis

In some embodiments additional measurements from additional sensors may be used to validate the training data. For example, an additional sensor (e.g., 130 of FIG. 1) such as a gyroscope may be used to measure subject movement. In this example, gyroscope data from each task was extracted from data streamed during testing, a 4-12 Hz bandpass filter applied to extract only tremor-related data, and, in some embodiments, additional methods (e.g., Welch's method) were applied to x, y, and z components individually. The area under the curve of each component may be approximated with the trapezoidal method and the sum of these values used as a ground truth for semi-instantaneous tremor severity assessment, denoted χ. Average level of tremor S, normalized for duration of test τ and thus defined by Equation 3, below:

$$s = \frac{\sum χ}{τ} \qquad Eqn . 3$$

The average level of tremor S, was derived for each test state. Tremor suppression J was defined as the fraction of tremor severity reduction as compared to tremor with stimulation Off, such that tremor suppression for a given control system may be defined with Equation 4, below:

$${J}_{system} = 1 - \frac{{S}_{system}}{{S}_{Off}} \qquad Eqn . 4$$

Quantifying Reduction in Energy Use

One advantage of using aDBS with a trained classifier may be a reduced energy drain, since stimulation is provided at an appropriate level to the subject's state. In this example embodiment, when the subject is determined to be at rest and the ‘Low’ stimulation procedure is selected, no stimulation is performed, saving drain on the battery of the IPG. Energy use per unit time was calculated using the total electrical energy delivered (TEED) methodology adapted to control for test duration T, such that

$${TEED}_{system} = \frac{{voltage}^{2} * frequency * pulse width}{impedence * τ} * seconds \qquad Eqn . 5$$

Energy use with aDBS may therefore be defined as the percentage of TEED saved in aDBS versus cDBS, such that relative energy saved Es is given by equation 6, below:

$${E}_{s} = ( 1 - \frac{{TEED}_{aDBS}}{{TEED}_{cDBS}} * ) 100 \% \qquad Eqn . 6$$

Methods for Evaluation of Therapeutic Accuracy

For the quantified portion of classifier evaluation, patients were asked to begin at rest with hands in their lap. At a semi-randomized time-stamped prompt, the patient was asked to conduct the finger-to-nose task of the FTM tremor rating scale continuously until the next prompt, at which point they were asked to return to rest. MU data was streamed continuously throughout the experiment, while stimulation amplitude was recorded on the Activa PC+S device during experiments and downloaded for analysis following the experiment.

A false positive ∈+ was said to occur when stimulation amplitude rose above ½ of a subject's clinically prescribed settings during a rest period, while a false negative ∈_ was defined as stimulation amplitude below this level during a period of movement. Total error rate ε was defined as the total number of errors divided by the duration of test τ, such that the total error is given by Equation 7, below:

$$ ε = \frac{\sum {ϵ}_{+} + \sum {ϵ}_{-}}{τ} \qquad Eqn . 7$$

This protocol was conducted with stimulation disabled, cDBS control system active, and aDBS control system active.

Following this controlled experimental protocol patients were asked to stand and move freely for some minutes in order to determine qualitatively whether they could detect a difference in the quality of their treatment or in the manifestation of side effects.

Therapeutic Performance During Controlled Testing

For testing data, it was determined that therapeutic classifier average total error rate was ε=0.468. However, over 92% of these errors were comprised of clinically permissible false positives; average false negative rate was ε_=0.036, indicating that stimulation was almost always being supplied at therapeutic levels during the experimental procedure. Use of the aDBS control system resulted in a 30.8% average drop in energy use by the neurostimulator. Further results may be found in table 2, below:

TABLE 2
Efficacy of aDBS:

| Patient | 1 | 2 |
|---|---|---|
| Percent energy saved | 34.0% | 27.5% |
| Overall error rate | 0.361 | 0.575 |
| False negative rate | 0.030 | 0.042 |
| cDBS tremor suppression | 0.596 | 0.125 |
| aDBS tremor suppression | 0.740 | 0.221 |

The extremely low false negative rates, paired with a significant reduction in overall stimulation, appear to have led to a substantial increase in tremor suppression in aDBS over cDBS, During their free movement period, subjects clearly and accurately differentiated between when stimulation was disabled and when some stimulation control system was active. However, they reported no differences in treatment efficacy between aDBS and cDBS,

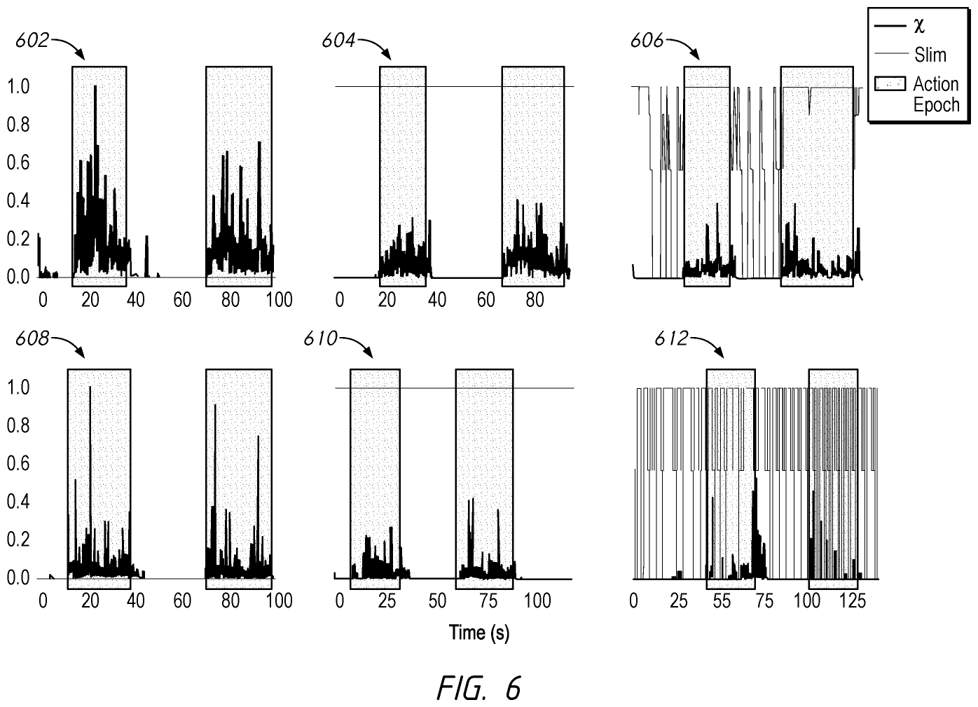

FIG. 6 is a set of graphs showing results of an implantable system using a classifier according to some embodiments of the present disclosure. The graphs 602 to 612 show the operation of an example implantable unit (e.g., 110 of FIG. 1) operating a classifier e.g., 122 of FIG. 1). In the example of FIG. 6, the classifier is the binary classifier trained using the training data as discussed in FIG. 4, with the biased classifier threshold of FIG. 5.

Each of the graphs 602 to 612 show stimulation amplitude, normalized by clinically determined maximum amplitude, and tremor severity χ, normalized to the maximum value in any state, with stimulation disabled, enabled and with aDBS active. The shaded background indicates patient was asked to perform finger-to-nose task, while blank background indicates patient instructed to rest with hands in lap. The graphs 602, 604, and 606 represent stimulation disabled, enabled, and with aDBS active respectively for P1, while the graphs 608, 610, and 612 show the same respective states for P2. In these situations, the ‘enabled’ state may mimic a cDBS system, where stimulation is continuously performed with no feedback from the implanted sensors.

The average tremor suppression across both patients with cDBS J_{cDBS}=0.361. Average tremor suppression with aDBS J_{aDBS}=0.481 for a 33.2% improvement with aDBS over cDBS, This is in line with previous findings that aDBS is more effective in tremor suppression than cDBS,

The versatility of the distributed training system (e.g., 100 of FIG. 1) may ensure relatively rapid training. For example, the entirety of the example procedure described herein, including explaining procedure and tests to subject, recording training data, reviewing data and repeating tests if needed, training classifier on selected data sets, and uploading classifier to patient device, was completed in under 20 minutes in each patient. This training time included repeat data collection for one state in each patient when initial data review revealed insufficient classification accuracy. Advantageously, this indicates that the brevity of the training process was due largely to the rapidity with which data could be analyzed and the ease of collecting more at-will with the distributed system descried herein.

Biasing the classifier towards the On state reduced overall accuracy; however, this may be a permissible condition. As may be seen in the analysis of training data, biasing resulted in marginal increases in overall error rate while cutting false negative rates by 28.7% for an overall sensitivity of 91.8%. In the analysis of therapeutic accuracy during testing, it is found that 96.4% of the time stimulation was required, it was provided. This increase in sensitivity may have to do with alteration of neural dynamics during stimulation ramping periods. The effect appears to be an increase in the likelihood of an On control signal.

In embodiments, the quantified analyses of symptom severity indicated that aDBS was substantially more effective in tremor suppression than cDBS on average.

That aDBS treatment was at least as effective as cDBS was supported by the patients' reports in their periods of free movement, during which they noticed no substantial differences in treatment efficacy. Paired with the quantitative results implying aDBS may be more effective than cDBS, this suggests that, while the differences in therapeutic efficacy may be below the threshold of perception for most patients already receiving cDBS, an aDBS system with an externally trained classifier may be able to operate with a lower maximum amplitude than that used in cDBS systems, thereby reducing overall stimulation to an even greater extent. This will further increase the already substantial energy savings seen by aDBS systems.

In some embodiments, specific patient programming may be performed to minimize, avoid, reverse, or stop various conditions experienced by patients or users of the distributed system, such as, for example, transient paresthesia. For example, a “maximum tolerable rate” test may be implemented during the training procedures in order to assist with patient comfort during aDBS,

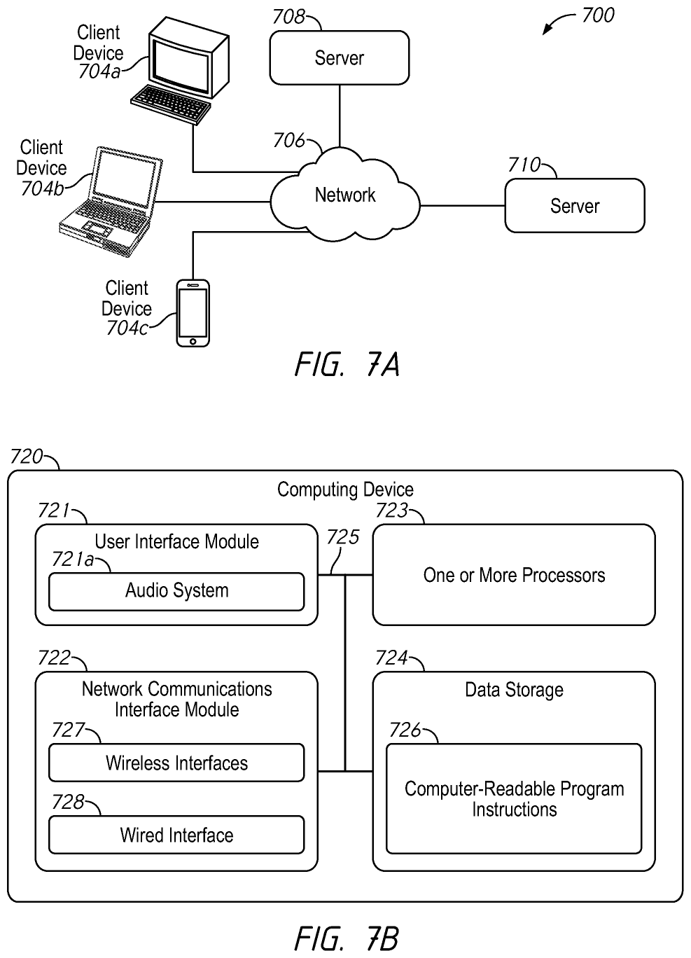

FIGS. 7A and 78 are block diagrams of an example computing network and computing device according to some embodiments of the present disclosure. The computing network 700 of FIG. 7A and/or the computing device 720 may, in some embodiments, be used to implement the implantable unit 110 and/or the external unit 150 of FIG. 1.

FIG. 7A is a block diagram of example computing network 700 in accordance with an example embodiment. In FIG. 7A, servers 708 and 710 are configured to communicate, via a network 706, with client devices 704a, 704h, and 704c. As shown in FIG. 7A, client devices can include a personal computer 704a, a laptop computer 704b, and a smartphone 704c. More generally, client devices 704a-704c (or any additional client devices) can be any sort of computing device, such as a workstation, network terminal, desktop computer, laptop computer, wireless communication device (e.g., a cell phone or smart phone), and so on. In particular, some or all of client devices 704a-704c can collect and process data associated with a neural data collection (such as, for example, a neurostimulator, a DBS probe, electrode strip, combinations thereof, or other suitable data collection and/or sensor devices or other types of client devices) as disclosed herein, as well as the device in which such neural stimulation is implemented or implemented in part. In many embodiments, client devices 704a-704c can perform most or all of the herein-described methods.

The network 706 can correspond to a local area network, a wide area network, a corporate intranet, the public Internet, combinations thereof, or any other type of network(s) configured to provide communication between networked computing devices. In some embodiments, part or all of the communication between networked computing devices can be secured.

Servers 708 and 710 can share content and/or provide content to client devices 704a-704c. As shown in FIG. 7A, servers 708 and 710 are not physically at the same location. Alternatively, servers 708 and 710 can be co-located, and/or can be accessible via a network separate from network 706. Although FIG. 7A shows three client devices and two servers, network 706 can service more or fewer than three client devices and/or more or fewer than two servers. In some embodiments, servers 708, 710 can perform some or all of the herein-described methods.

FIG. 7B is a block diagram of an example computing device 720 including user interface module 721, network-communication interface module 722, one or more processors 723, and data storage 724, in accordance with embodiments of the invention.

In particular, computing device 720 shown in FIG. 7A can be configured to perform one or more functions of a system, client devices 704a-704c, network 706, and/or servers 708, 710. Computing device 720 may include a user interface module 721, a network-communication interface module 722, one or more processors 723, and data storage 724, all of which may be linked together via a system bus, network, or other connection mechanism 725.

Computing device 720 can be a desktop computer, laptop or notebook computer, personal data assistant (PDA), mobile phone, video game console, embedded processor, touchless-enabled device, or any similar device that is equipped with at least one processing unit capable of executing machine-language instructions that implement at least part of the herein-described tremor suppression techniques and methods. In many embodiments, computing device 720 may be implemented using a smartphone.

User interface 721 can receive input and/or provide output, perhaps to a user. User interface 721 can be configured to send and/or receive data to and/or from user input from input device(s), such as a microphone, a keyboard, a keypad, a touch screen, a computer mouse, a track ball, a joystick, camera, and/or other similar devices configured to receive input from a user of the computing device 720. In some embodiments, input devices can include gesture-related devices, such a video input device, a motion input device, time-of-flight sensor, RGB camera, or other 3D input device. User interface 721 can be configured to provide output to output display devices, such as one or more cathode ray tubes (CRTs), liquid crystal displays (LCDs), light emitting diodes (LEDs), displays using digital light processing (DU′) technology, printers, light bulbs, and/or other similar devices capable of displaying graphical, textual, and/or numerical information to a user of computing device 720. User interface module 721 can also be configured to generate audible output(s), such as a speaker, speaker jack, audio output port, audio output device, earphones, and/or other similar devices configured to convey sound and/or audible information to a user of computing device 720.

Network-communication interface module 722 can be configured to send and receive data over wireless interface 727 and/or wired interface 728 via a network, such as network 706. Wireless interface 727 if present, can utilize an air interface, such as a Bluetooth®, Wi-Fi®, ZigBee®, and/or WiMAX™ interface to a data network, such as a wide area network (WAN), a local area network (LAN), one or more public data networks (e.g., the Internet), one or more private data networks, or any combination of public and private data networks. Wired interface(s) 728, if present, can comprise a wire, cable, fiber-optic link and/or similar physical connection(s) to a data network, such as a WAN, LAN, one or more public data networks, one or more private data networks, or any combination of such networks.

In some embodiments, network-communication interface module 722 can be configured to provide reliable, secured, and/or authenticated communications. Communications can be made secure (e.g., be encoded or encrypted) and/or decrypted/decoded using one or more cryptographic protocols and/or algorithms, such as, but not limited to, DES, AES, RSA, Diffie-Hellman, and/or DSA. Other cryptographic protocols and/or algorithms can be used as well as or in addition to those listed herein to secure (and then decrypt/decode) communications.

Processor(s) 723 can include one or more central processing units, computer processors, mobile processors, digital signal processors (DSPs), microprocessors, computer chips, and/or other processing units configured to execute machine-language instructions and process data. Processor(s) 723 can be configured to execute computer-readable program instructions 726 that are contained in data storage 724 and/or other instructions as described herein.

Data storage 724 can include one or more physical and/or non-transitory storage devices, such as read-only memory (ROM), random access memory (RAM), removable-disk-drive memory, hard-disk memory, magnetic-tape memory, flash memory, and/or other storage devices. Data storage 724 can include one or more physical and/or non-transitory storage devices with at least enough combined storage capacity to contain computer-readable program instructions 726 and any associated/related data structures.

Computer-readable program instructions 726 and any data structures contained in data storage 726 include computer-readable program instructions executable by processor(s) 723 and any storage required, respectively, to perform at least part of herein-described methods for tremor suppression using the embedded aDBS system described herein.

Of course, it is to be appreciated that any one of the examples, embodiments or processes described herein may be combined with one or more other examples, embodiments and/or processes or be separated and/or performed amongst separate devices or device portions in accordance with the present systems, devices and methods.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

As used herein and unless otherwise indicated, the terms “a” and “an” are taken to mean “one”, “at least one” or “one or more”. Unless otherwise required by context, singular terms used herein shall include pluralities and plural terms shall include the singular.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above,” and “below” and words of similar import, when used in this application, shall refer to this application as a whole and not to any particular portions of the application.

The description of embodiments of the disclosure is not intended to be exhaustive or to limit the disclosure to the precise form disclosed. While the specific embodiments of, and examples for, the disclosure are described herein for illustrative purposes, various equivalent modifications are possible within the scope of the disclosure, as those skilled in the relevant art will recognize.

Specific elements of any foregoing embodiments can be combined or substituted for elements in other embodiments. Moreover, the inclusion of specific elements in at least some of these embodiments may be optional, wherein further embodiments may include one or more embodiments that specifically exclude one or more of these specific elements. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

Finally, the above-discussion is intended to be merely illustrative of the present system and should not be construed as limiting the appended claims to any particular embodiment or group of embodiments. Thus, while the present system has been described in particular detail with reference to exemplary embodiments, it should also be appreciated that numerous modifications and alternative embodiments may be devised by those having ordinary skill in the art without departing from the broader and intended spirit and scope of the present system as set forth in the claims that follow. Accordingly, the specification and drawings are to be regarded in an illustrative manner and are not intended to limit the scope of the appended claims.

What is claimed is:

1. A method comprising:
receiving sensor data from implanted sensors at an external unit;
classifying the sensor data based on symptom information;
training a machine learning model to generate a classifier based on the classified sensor data; and
loading the classifier onto an implantable unit.

2. The method of claim 1, further comprising:
selecting a stimulation procedure of the implantable unit based on the sensor data from the implanted sensors and the classifier; and
providing stimulation to a subject from the implantable unit based on the selected stimulation procedure.

3. The method of claim 2, further comprising:
selecting a first stimulation procedure based on a first result from the classifier; and
selecting a second stimulation procedure based on a second result from the classifier.

4. The method of claim 1, further comprising obtaining the symptom information from an additional sensor.

5. The method of claim 4, wherein the additional sensor is placed externally on the subject.

6. The method of claim 1, wherein the external unit includes one or more networked devices in a cloud computing system.

7. The method of claim 1, further comprising:
collecting a first set of sensor data while the subject is at rest, and a second set of sensor data while the patient is active; and
training the classifier to determine if the subject is at rest or if the subject is active based on the first set of sensor data and the second set of sensor data.

8. The method of claim 7, wherein the first set of sensor data includes a first portion where the implantable unit is providing active stimulation and a second portion where the implantable unit is not providing active stimulation and wherein the second set of sensor data includes a third portion where the implantable unit is providing active stimulation and a fourth portion where the implantable unit is not providing active stimulation.

9. The method of claim 1, further comprising biasing the classifier.

10. The method of claim 1, wherein the sensor data includes information from implantable sensors and from a stimulation electrode.

11. A system comprising:
an implantable unit implanted in a subject, the implantable unit comprising:
implanted sensors configured to provide sensor information;
a stimulation electrode;
a processor; and
a memory loaded with non-transitory instructions, which when executed by the processor cause the implantable unit to:
select a stimulation procedure based on the sensor information and a classifier; and
apply stimulation to the stimulation electrode based on the selected stimulation procedure; and
an external unit comprising:
a processor; and
a memory loaded with non-transitory instructions, which when executed by the processor cause the external unit to:
train the classifier based on data from the sensors and symptom information; and
load the classifier onto the memory of the implantable unit.

12. The system of claim 11, wherein the implantable unit is an adaptive deep brain stimulation (aDBS) system.

13. The system of claim 11, wherein the implantable sensors include electrocorticography (ECoG) strips configured to collect local field potential (LFP) information.

14. The system of claim 11, wherein the memory of the external unit includes instructions which, when executed by the processor of the external unit, cause the external unit to train the classifier to determine active or at rest state of subject.

15. The system of claim 14, wherein the memory of the implantable unit includes instructions which, when executed by the processor of the implantable unit, cause the implantable unit to select a first stimulation procedure when the classifier determines that the subject is active and select a second stimulation procedure when the classifier determines that the subject is at the rest state.

16. The system of claim 14, wherein the classifier is biased to preferentially select the active state based on the sensor information.

17. The system of claim 11, wherein the symptom information includes labels for sensor information collected during different periods of subject activity.

18. The system of claim 11, wherein the external unit includes one or more networked systems in a location remote from the implantable unit.

19. The system of claim 11, wherein the stimulation electrode is a deep brain stimulation electrode implanted in the subject's nervous system.

20. The system of claim 11, further comprising a wearable sensor placed on the subject, wherein the symptom information is based, in part, on information from the wearable sensor.

21. An apparatus comprising:
implanted sensors configured to provide sensor information;
a stimulation electrode;
an implantable unit configured to classify the sensor information based on a classifier, select a stimulation procedure based on the classified sensor information and provide stimulation via the stimulation electrode based on a selected stimulation procedure, wherein the classifier is trained by a machine learning algorithm, and wherein the implantable unit is configured to apply stimulation with the stimulation electrode based on the selected stimulation procedure.

22. The apparatus of claim 21, wherein the classifier is trained on an external unit which is not implanted in the subject.

23. The apparatus of claim 22, wherein the classifier is trained based on the sensor information from the implantable sensors and information from the stimulation electrode.

24. The apparatus of claim 21, wherein the implanted sensors include an electrocorticography (ECoG) strip.

25. The apparatus of claim 21, wherein the classifier is configured to determine if a subject is at an active state or a rest state.

26. The apparatus of claim 25, wherein the implantable unit is configured to provide stimulation with the stimulation electrode when the classifier determines the active state and configured to not provide stimulation with the stimulation electrode when the classifier determines the rest state.

27. The apparatus of claim 21, wherein the implanted sensors, the stimulation electrode, and the implantable unit are components of an adaptive deep brain stimulation (aDBS) system.
